(19) United States
(12) Patent Application Publication
(10) Pub. No.: US 2024/0325297 A1
(43) Pub. Date: Oct. 3, 2024

(54) ELECTROLYTE HYDRATION COMPOSITIONS AND METHODS OF MAKING AND USING THEREOF

(71) Applicant: Connie Wan, Renton, WA (US)

(72) Inventor: Connie Wan, Renton, WA (US)

(21) Appl. No.: 18/619,155

(22) Filed: Mar. 27, 2024

Related U.S. Application Data
(60) Provisional application No. 63/454,723, filed on Mar. 27, 2023.

(51) Int. Cl. A61K 9/00; A61K 33/08; A61K 33/14; A61K 33/30; A61K 36/752; A61K 36/889; A61K 47/36

(52) U.S. Cl. CPC A61K 9/0056; A61K 33/08; A61K 33/14; A61K 33/30; A61K 36/752; A61K 36/889; A61K 47/36

(57) ABSTRACT

An electrolyte hydration composition, comprising, a mineral component, wherein the mineral component comprises salts of sodium, potassium, magnesium, or zinc, a carbohydrate composition, wherein the carbohydrate composition comprises a high glycemic index (GI) sugar having a GI not less than 70, and a gelling component in a sufficient amount to provide a cohesive gelled product.

### CROSS-REFERENCES TO RELATED APPLICATIONS

This application claims the benefit of priority from, and hereby incorporates by reference the entire disclosure, co-pending U.S. Provisional Application for Patent Ser. No. 63/454,723, filed Mar. 27, 2023.

### TECHNICAL FIELD

The application relates generally to high-loading electrolytes or minerals composition in the chewable gel or gummy formula and methods of administration of various gummy or chewable gel compositions for supporting hydration.

### BACKGROUND

Electrolytes are minerals found in the blood that help regulate and control the balance of fluids in the body. These minerals play a role in keeping the biological system functioning properly including, for example, regulating blood pressure, regulating muscle contraction, and keeping vital organs functioning.

The electrolyte balance is needed for optimal health and physical performance. Electrolyte balance can be disturbed through intense exercise, sweating, vomiting, or diarrhea. For example, intense exercise can cause electrolyte loss through sweating. Fever-caused excessive sweating can also lead to electrolyte loss. Vomiting and diarrhea can cause significant fluid as well as electrolyte loss. Losing a significant number of electrolytes can cause dehydration, muscle cramping, and spasms.

There is a need for a fast, effective, and convenient way to deliver electrolytes into the body.

### SUMMARY

The application provides electrolyte hydration compositions to provide or support electrolyte balance and promote hydration. In one embodiment, the electrolyte hydration composition comprises a mineral composition, a carbohydrate composition, and a gelling component in a sufficient amount to provide a cohesive gelled product.

In one embodiment, the mineral composition comprises salts of sodium, potassium, magnesium, zinc, or a combination thereof. In one embodiment, the mineral composition may further include calcium, iron, copper, molybdenum, manganese, phosphorus, iodine, nickel, cobalt, or selenium.

The salt may be ionic or chelated salt form. The ionic salt may be any conventional salt including without limitation salts of halogen ions (including for example chloride, iodide, etc.), sulfates, phosphates, or carbonates. The chelated salt may be amino acid chelate. In one embodiment, the salt may be glycinate, lactate, or gluconate.

The salt may be salt derived from inorganic acid or organic acid. Examples of inorganic acids include HCl, HBr, HI, H2SO4, H3PO4, or other similar inorganic acids that are known in the chemistry art. Examples of organic acids include citric acid, lactic acid, tartaric acid, acetic acid, propanoic acid, malic acid, maleic acid, pantothenic acid, galactic acid, fatty acids (such as palmitic acid, lauric acid, stearic acid, etc.), amino acids, 2-aminoethanesulfonic acid (taurine), or other similar organic acids that are known in the chemistry art.

The salt of sodium may include sodium chloride, sodium iodide, sodium citrate, trisodium citrate dihydrate, sodium gluconate, sodium bicarbonate, sodium carbonate, sodium alginate, or salts of phosphate. The salt of potassium may include potassium chloride, potassium iodide, potassium citrate, potassium gluconate, potassium bicarbonate, potassium carbonate, or potassium salts of phosphate. The salt of magnesium may include magnesium oxide, magnesium glycinate, magnesium citrate, magnesium malate, magnesium taurate, magnesium acetyl taurate, magnesium theronate, or salts of phosphate. The salt of zinc may include zinc citrate, zinc picolinate, zinc oxide, zinc carnosine, zinc picolinate or monomethionate, or salts of phosphate. The salt of calcium may include calcium gluconate, calcium carbonate, calcium chloride, salts of phosphate, or pantothenate.

In one embodiment, the electrolyte hydration composition comprises at least 10%, 9%, 8%, 7%, 6%, 5%, 4%, or 3% of the mineral composition. In one embodiment, the electrolyte hydration composition comprises from about 2% to about 6%, from about 1.8% to about 5.2%, from about 2.1% to about 5%, from about 2.3% to about 4%, from about 3% to about 4%, from about 2.8% to about 4.2%, from about 3% to about 5% of the mineral composition.

In one embodiment, the electrolyte hydration composition comprises at least 2%, 2.5%, 3%, 4%, or 5%, 6%, 7%, 8%, 9% or 10% of sodium salt. In one embodiment, the electrolyte hydration composition comprises at least 2.1%, 2.2%, 2.3%, 2.4%, 2.5%, 2.6%, 2.7%, or 3% of sodium salt. In one embodiment, the electrolyte hydration composition comprises from about 2% to about 6%, from about 1.8% to about 5.2%, from about 2.1% to about 5%, from about 2.3% to about 4%, from about 3% to about 4%, from about 2.8% to about 4.2%, from about 3% to about 5% of the sodium salt.

In one embodiment, the electrolyte hydration composition comprises at least 0.5%, 1%, 2%, 2.5%, 3%, 4%, or 5%, 6%, 7%, or 8% of potassium salt. In one embodiment, the electrolyte hydration composition comprises at least 0.5%, 1%, 2%, 2.1%, 2.2%, 2.3%, 2.4%, 2.5%, 2.6%, 2.7%, or 3% of potassium salt. In one embodiment, the electrolyte hydration composition comprises from about 0.5% to about 3%, from about 0.7% to about 3.2%, from about 0.5% to about 2.8%, from about 0.8% to about 4%, from about 2% to about 4%, from about 1% to about 7%, from about 2% to about 8% of the potassium salt.

In one embodiment, the electrolyte hydration composition comprises at least 0.08%, 0.1%, 0.2%, 1%, 2%, 2.5%, 3%, 4%, 5%, or 6% of zinc salt. In one embodiment, the electrolyte hydration composition comprises at least 0.05%, 0.07%, 0.08%, 1%, 2%, 2.1%, 2.2%, 2.3%, 2.4%, 2.5%, 2.6%, 2.7%, or 3% of zinc salt. In one embodiment, the electrolyte hydration composition comprises from about 0.05% to about 0.3%, from about 0.07% to about 1%, from about 0.09% to about 2.8%, from about 0.1% to about 4%, from about 1.5% to about 6%, from about 1% to about 5%, from about 1.2% to about 6% of the zinc salt.

In one embodiment, the electrolyte hydration composition comprises at least 0.1%, 0.2%, 1%, 2%, 2.5%, 3%, 4%, 5%, 6%, 8%, 10%, or 12% of magnesium salt. In one embodiment, the electrolyte hydration composition comprises at least 0.12%, 1%, 1.5%, 2.1%, 2.4%, 2.5%, 2.6%, or 3% of magnesium salt. In one embodiment, the electrolyte hydration composition comprises from about 0.1% to about 0.3%, from about 0.15% to about 1%, from about 0.2% to about 2.8%, from about 0.25% to about 4%, from about 1% to about 6%, from about 2% to about 5%, from about 3% to about 8% of the magnesium salt.

In one embodiment, the electrolyte hydration composition comprises at least 0.1%, 0.2%, 1%, 2%, 2.5%, 3%, 4%, 5%, 6%, 8%, 10%, or 12% of calcium salt. Not wanting to be limited by theory, through extensive experimentation, it was discovered that a concentration of Calcium salts in the amount of at least 0.05% significantly increases the structure integrity of the high-loading electrolyte gummy composition. In one embodiment, the electrolyte hydration composition comprises at least 0.12%, 0.14%, 0.15%, 0.2%, 0.3%, 0.4%, 0.5%, 1%, 1.5%, 2.1%, 2.4%, 2.5%, 2.6%, or 3% of calcium salt. In one embodiment, the electrolyte hydration composition comprises from about 0.1% to about 0.3%, from about 0.15% to about 1%, from about 0.2% to about 2.8%, from about 0.25% to about 4%, from about 1% to about 6%, from about 2% to about 5%, from about 3% to about 8% of the calcium salt.

In one embodiment, the electrolyte hydration composition comprises from about 54% to about 89%, about 56% to about 81%, about 60% to about 78%, about 68% to about 76%, about 62% to about 73%, about70% to about 75%, about 67% to about 75% of the carbohydrate composition. In one embodiment. the electrolyte hydration composition comprises about 67%, 68%, 71%, 71%, 75%, or 76% of carbohydrate composition.

The carbohydrate composition comprises polysaccharides or polysaccharide sugar alcohol, disaccharides or disaccharide sugar alcohol, and monosaccharides or monosaccharides sugar alcohol. In one embodiment, the electrolyte hydration composition may include monosaccharides or monosaccharides sugar alcohol in an amount from about 7% to about 25%, from about 5% to about 11%, from 7% to about 10%, or from about 20% to about 25%. In one embodiment, the electrolyte hydration composition may include disaccharides or disaccharide sugar alcohol in an amount from about 30% to about 68%, from about 25% to about 62%, from about 35% to about 55%, or from about 40% to about 57%. In one embodiment, the electrolyte hydration composition may include polysaccharides or polysaccharide sugar alcohol from about 2% to about 30%, from about 6% to about 25%, from about 8% to about 15%, or from about 9% to about 20%. The ratio of the amounts of monosaccharides, disaccharides, and polysaccharides.

The polysaccharides may include a glucose syrup from any plant source. In one embodiment, the glucose syrup may be tapioca syrup, corn syrup, rice syrup, yam syrup, sorghum syrup, or wheat syrup. In one embodiment, the glucose syrup may include not more than 10%, 20%, 24%, 40%, 42%, 60%, or 80% of monosaccharides. In one embodiment, the glucose syrup has a monosaccharide content of not more than 20%, 22%, 24%, or 26%. In one embodiment, the glucose syrup has a monosaccharide content of not more than 40%, 41%, 42%, 43%, or 45%. The glycose syrup may be a liquid or solid having a DE of greater than 42, e.g., 63 DE. The higher DE values will decrease the tendency for granulation and aid in maintaining softness. In one embodiment, the glucose syrup is a DE24 or DE40 tapioca syrup. In one embodiment, the glucose syrup is a DE24 or DE42 corn syrup. In one embodiment, the glucose syrup is a high maltose DE24 or DE 40 sorghum syrup. In one embodiment, the monosaccharide comprises glucose.

In one embodiment, the polysaccharides may be panose, isomaltotriose, isomaltopentose, maltodextrin, starch, resistant starch, polydextrin, fructooligosaccharides, inulin, galactooligosaccharides, xylooligosaccharides, isomaltooligosaccharides, lactulose, or a combination thereof. In one embodiment, polysaccharides may be a soluble dietary fiber.

The disaccharides may include sucrose, lactose, maltose, trehalose, isomaltose, isomaltulose, lactulose, sophorose, cellobiose, melibiose, gentiobiose, nigerose, or laminaribiose.

The monosaccharides may include glucose, fructose, galactose, ribose, deoxyribose, mannose, xylose, arabinose, allulose, or a combination thereof.

In one embodiment, the carbohydrate composition comprises a high glycemic index (GI) sugar having a GI not less than 170, 160, 140, 120, 110, 100, 90, 80, or 70. In one embodiment, the carbohydrates have a low glycemic index (GI) sugar having a GI of not more than 8, 9, 10, 15, 20, 25 or 30. In one embodiment, the carbohydrates have a low glycemic index (GI) sugar having a GI of 0. In one embodiment, the zero glycemic index sugar comprises allulose (psicose).

In one embodiment, the electrolyte hydration composition has a GI of not less than 140, 120, 110, 100, 90, 80, or 70. In one embodiment, the electrolyte hydration composition has a GI of not more than 8, 9, 10, 15, 20, 25 or 30. In one embodiment, the electrolyte hydration composition has a GI of 0.

In one embodiment, the carbohydrate composition comprises sugar alcohol. In one embodiment, the electrolyte hydration composition is sugar-free. In one embodiment, the electrolyte hydration composition is substantially free of sucrose, glucose, or fructose.

In one embodiment, the carbohydrate composition comprises monosaccharide sugar alcohol, disaccharide sugar alcohol, and polysaccharide sugar alcohol. The polysaccharide sugar alcohol comprises long-chain polyhydroxy alcohols. The disaccharides sugar alcohol comprises maltulose, isomaltulose, maltitol, isomaltitol, isomalt, lactitol, or a combination thereof. The monosaccharides sugar alcohol comprises sorbitol, mannitol, xylitol, erythritol, arabitol, ribitol, glycerol, galactitol, sorbitan, iditol, or a combination thereof.

The carbohydrate composition may further comprise glucosamine, galactosamine, or a combination thereof.

The electrolyte hydration composition may further include a vitamin composition, an amino acid composition, an antioxidant composition, an anti-inflammatory composition, or an herb composition.

In one embodiment, the vitamin composition comprises vitamins A, B, C, D, E, K or a combination thereof. In one embodiment, the electrolyte hydration composition further comprises Vitamin B composition.

In one embodiment, the Vitamin B composition comprises Vitamin B1 (thiamine), Vitamin B2 (riboflavin), niacin, vitamin B6, vitamin B12, folate, pantothenic acid, biotin, or derivatives thereof. In one embodiment, the vitamin B composition comprises niacin, Vitamin B2, Vitamin B6, and Vitamin B12.

In one embodiment, the amino acid composition comprises at least one essential amino acid or its derivative thereof. In one embodiment, the amino acid composition comprises a mix of BCAA.

In one embodiment, the antioxidant composition comprises bioflavonoids, vitamin E, vitamin C, resveratrol, coenzyme Q10, quercetin, rutin, lycopene, L-glutathione, N-acetyl cysteine, phenolics, anthocyanins, flavonoids, anthracenes, carotenoids, zeaxanthin, astaxanthin, xanthin, pomegranate, ginkgo biloba, green tea, garlic, grapeseed, blackberry, elderberry, cranberry, blueberry, saffron, Sangre de grado (dragon's blood), lyceum barbarum (Gouqizi), beet, its extract, powder, or isolates thereof.

In one embodiment, the anti-inflammatory composition comprises acetylsalicylic acid, bioflavonoids, omega-3 fatty acids, white willow, curcumin, turmeric, saffron, ginger, Boswellia, chamomile, licorice, arnica, witch hazel, Echinacea, bromelain from pineapple, yarrow, oak bark, walnut leaf, fenugreek, aloe vera, calendula, capsaicin, mallow, wild pansy, an extract thereof, powder thereof, or a combination thereof.

In one embodiment, the electrolyte hydration composition comprises from about 1% to about 60%, about 20% to about 50%, about 3.2% to about 4.5%, about 6% to about 10%, about 2.7% to about 4.8%, about 10% to about 30%, about 3.5% to about 5% of the gelling composition. In one embodiment, the electrolyte hydration composition comprises about 3%, 3.5%, 3.7%, 3.9%, 4.2%, 4.6%, 5%, 6%, or 7.9% of carbohydrate composition.

In one embodiment, the gelling component comprises gelatin, starch, pectin, gellan gum, gum Arabic, carrageenans, guar, agar, alginate, locust bean gum, xanthan, or derivatives thereof. In one embodiment, the starch comprises corn starch, tapioca starch, potato starch, wheat starch, thin-boiling starch, high amylose corn starch, instant starches, stick rice starch, or derivatives thereof. In one embodiment, the electrolyte hydration composition is essentially free of gelatin.

In one embodiment, the electrolyte hydration composition comprises about 2.9%, 3.1%, 3.5%, 3.7%, 3.9%, 4.1%, 4.5% of pectin as gelling agent. In one embodiment, the electrolyte hydration composition comprises about 6.2%, 6.8%, 7.3%, 7.9%, 8.1%, 9%, 9.5%, 10%, 10.2%, 11% or 12% of gelatin as the gelling agent. In one embodiment, the electrolyte hydration composition comprises about 9.5%, 10.2%, 12%, 15%, 19%, 17%, 18%, 20%, 25%, 29% or 33% of starch as the gelling agent. In one embodiment, the electrolyte hydration composition comprises from about 1% to about 4% of agar as the gelling agent. In one embodiment, the electrolyte hydration composition comprises from about 18% to about 54% of gum Arabic as the gelling agent.

In one embodiment, the electrolyte hydration composition further comprises an additive selected from sweeteners, food acids, flavoring agents, coloring agents, humectants, bulking agents, fatty acids, triglycerides, plasticizers, emulsifiers, thickeners, preservatives, or and a mixture thereof.

In one embodiment, the composition may include sweeteners including, for example, sugar, glucose syrup, corn syrup, high fructose corn syrup, juice concentrate, or mixtures thereof. In one embodiment, the sweetener comprises erythritol, xylitol, sugar, glucose syrup, corn syrup, high fructose corn syrup, juice concentrate, tapioca syrup, agave syrup, brown rice syrup, high maltose syrup, invert sugar, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N-[N-[3-(3-hydroxy-4-methoxyphenyl) propyl]-L-.alpha.-aspartyl]-L-phenylalanine 1-methyl ester, N-[N-[3-(3-hydroxy-4-methoxyphenyl)-3-methylbutyl]-L-alpha-aspartyl]-L-phenylalanine 1-methyl ester, N-[N-[3-(3-methoxy-4-hydroxyphenyl) propyl]-L-alpha-aspartyl]-L-phenylal-anine 1-methyl ester, salts thereof, licorice or its extracts or isolates, or a mixture thereof.

The pH of the hydration composition may be adjusted by the addition of suitable food acid, buffer, or both. The pH of the composition may be from about 3 to about 4.5, more preferably about 3.2 to about 4, about 3.2 to about 3.5. In one embodiment, the pH is 3.2, 3.4, or 3.5.

In one embodiment, the food acid comprises citric acid, malic acid, fumaric acid, lactic acid, tartaric acid, gluconic-delta lactone, salts of gluconic acid, phosphoric acid, succinic acid, adipic acid, acetic acid, or a combination thereof.

In one embodiment, the flavoring agent can be any flavoring extract or oils from fruits, plants, flowers, or synthetic. Examples include vanilla, peppermint oil, spearmint oil, eucalyptus oil, cinnamon oil, grapefruit oil, orange oil, pineapple oil, citrus oils, fruit essences, hibiscus, or strawberry extract.

In one embodiment, the bulking agent comprises maltitol, polydextrose, sorbitol, soluble corn fiber, resistant starch, resistant maltodextrin, cellulose, hemicellulose, fructo-oligosaccharides, galacto-oligosaccharides, lactulose, xylo-isomalto-oligosaccharide, soybean oligosaccharide, oligo-glucose, stachyose, lactosucrose, or a combination thereof.
In another aspect, the application provides methods for making the electrolyte hydration composition disclosed thereof. In one embodiment, the method includes the steps of: preparing a first premix by heating a mixture of ingredients comprising the carbohydrate composition, the mineral composition, and water sufficiently to dissolve in the water; preparing a gelling solution by heating a gelling composition, a carbohydrate composition in water sufficiently to dissolve in.

These and other objects and advantages of the application shall become apparent from the detailed description that follows.

### DETAILED DESCRIPTION

The application provides electrolyte hydration compositions to promote electrolyte balance and hydration.

In one embodiment, the electrolyte hydration composition includes a gelling component in a sufficient amount to provide a cohesive gelled product, a mineral composition, and a carbohydrate composition.

In one embodiment, the mineral composition comprises salts or chelates of sodium, potassium, magnesium, zinc, calcium, iron, chloride, copper, molybdenum, manganese, phosphorus, iodine, nickel, or selenium, selenium yeast, or a combination thereof. The minerals may be in the forms of salts or chelates. In one embodiment, the chelates may be amino acid chelates or peptide chelates.

In one embodiment, the mineral composition comprises sodium and potassium. In one embodiment, the mineral composition comprises essentially sodium, potassium, magnesium, and zinc.

In one embodiment, the electrolyte hydration composition comprises at least 1.5%, 2%, 2.1%, 2.2%, or 2.5% of NaCl, or at least 0.75%, 0.8%, 0.9% or 1% of sodium ion by weight.

In one embodiment, the electrolyte hydration composition comprises at least 0.5%, 0.6%, 0.7%, 0.75%, or 0.8% of KCl, or at least 0.3%, 0.4%, 0.45% or 0.5% of potassium ion by weight.

In some embodiments, the electrolyte hydration composition may from about 0.5% to about 3.5%, from about 0.7% to about 3.2%, from about 2% to about 3.5%, or from about 1% to about 4% by weight of the mineral composition.

The high GI sugar may have a GI at least 70, 80, 90, 100, 110, 120, 150, 170, 200, or higher. In one embodiment, the high GI sugar comprises glucose, maltose, dextrose, maltodextrin, starch, glucose syrup, or a combination thereof.

In one embodiment, the electrolyte hydration composition comprises, about 55%-80%, 60%-72%, or 65%-75% by weight by weight of the carbohydrate composition.

The electrolyte hydration composition may further comprise a vitamin composition, an amino acid composition, an antioxidant composition, an herbal composition, or an anti-inflammatory composition.

In some embodiments, the vitamin composition may include fat-soluble vitamins such as vitamin A, vitamin D, vitamin E, and vitamin K and combinations thereof.

In some embodiments, the composition includes a vitamin B composition. In one embodiment, the vitamin B composition comprises niacin, Vitamin B2, Vitamin B6, and Vitamin B12. In some embodiments, the Vitamin B composition consists of folate, B6, and B12. In some embodiments, the vitamin B composition consists of thiamine (B1), riboflavin (B2), niacin (B3), pantothenic acid (B5), pyridoxine (B6), biotin (B7), folic acid (B9), cobalamins (B12), and derivatives or combinations thereof.

In one embodiment, the electrolyte hydration composition further comprises an amino acid composition. In one embodiment, the amino acid composition comprises at least one essential amino acid. In one embodiment, the amino acid composition comprises phenylalanine, valine, threonine, tryptophan, methionine, leucine, isoleucine, lysine, and histidine. In one embodiment, the amino acid composition comprises leucine, isoleucine, and valine.

In some embodiments, the electrolyte hydration composition includes an antioxidant composition. In some embodiments, the antioxidant composition can include but are not limited to ascorbic acid, citric acid, rosemary oil, vitamin A, vitamin E, vitamin E phosphate, tocopherols, di-alpha-tocopherol phosphate, tocotrienols, coenzyme Q10, alpha lipoic acid, dihydrolipoic acid, xanthophylls, beta-cryptoxanthin, lycopene, lutein, zeaxanthin, astaxanthin, beta-carotene, carotenes, mixed carotenoids, polyphenols, flavonoids, and combinations thereof. In some embodiments, the antioxidant composition may include extracts and active phytochemicals such as ferulic acid (from apples), ginseng, ginkgo biloba, beta carotene, capsicanoids, anthocyanidins, bioflavonoids, d-limonene, isothiocyanates, ginger, grapes, catechins and polyphenols from teas, phytosterols, isoflavones, lycopene, curcumin, glucosamine, pomegranate extract, resveratrol, quercetin, rutin, lycopene, glutathione (GSH), N-Acetylcysteine (NAC)

The electrolyte hydration composition may further include anti-inflammatory composition. The anti-inflammatory composition includes an agent that is capable of reducing or attenuating inflammation. In one embodiment, the anti-inflammatory composition comprises acetylsalicylic acid, bioflavonoids, omega-3 fatty acids, white willow, curcumin, turmeric, saffron, ginger, Boswellia, chamomile, licorice, arnica, witch hazel, Echinacea, bromelain from pineapple, yarrow, oak bark, walnut leaf, fenugreek, aloe vera, calendula, capsaicin, mallow, wild pansy, an extract thereof, powder thereof, or a combination thereof. In one embodiment, the anti-inflammatory composition comprises acetylsalicylic acid, willow or its extract, curcumin, ginger or its extract, or a combination thereof. In one embodiment, the anti-inflammatory composition consists essentially of acetylsalicylic acid, curcumin, and ginger root extract.

In some embodiments, the electrolyte hydration composition may further include sweeteners, food acids, flavoring agents, coloring agents, sensate agents, freshening agents, probiotics, prebiotics, bulking agents, humectants, plasticizers, preservatives, stabilizing agents, emulsifiers, or thickening agents.

The electrolyte hydration composition may further include fibers or prebiotics. Any suitable fiber can be used. In one embodiment, a naturally derived fiber is used, including one or more selected from naturally derived inulin, inulin extract, synthetic inulin, hydrolysis products of inulin commonly known as fructooligosaccharides, galactooligosaccharides, xylooligosaccharides, oligo derivatives of starch, husks, brans, psyllium, polysaccharides, starches, polycarbophil, lignin, arabinogalactans, chitosan, oat fiber, soluble corn fiber, non-digestible corn dextrin, non-digestible wheat dextrin, locust bean gum and derivatives of locust bean gum, hydroxypropylmethylcellulose (HPMC), pectin, and mixtures thereof. In some embodiments, fibers may include inulin, wheat dextrin, or fructooligosaccharides.

### Sweetener

In general, an effective amount of sweetener may be utilized to provide the level of sweetness desired, and this amount may vary with the sweetener selected. In some embodiments, the amount of sweetener may be present in amounts from about 0.001% to about 3%, by weight of the composition, depending upon the sweetener or combination of sweeteners used. The exact range of amounts for each type of sweetener may be selected by those skilled in the art.

Sweeteners may include one or more monosaccharides or disaccharides. Examples include sugar, sucrose, invert sugar, dextrose, lactose, honey, malt syrup, malt syrup solids, maltose, fructose, granular fructose, maple syrup, rice syrup, rice syrup solids, sorghum syrup, refiners syrup, corn syrup, corn syrup solids, high fructose corn syrup, molasses, or combinations thereof.

In one embodiment, the sweetener include common sugars such as sucrose and glucose, polyols such as maltitol, erythritol, and isomalt, syrup sweeteners such as glucose syrup, corn syrup, high fructose corn syrup, and juice concentrates.

In one embodiment, artificial sweeteners can be used such as acesulfame K, aspartame, sucralose, d-tagatose, neotame, monatin, and acesulfame potassium (Ace-K), or combinations thereof.

The sweeteners involved may be selected from a wide range of materials including water-soluble sweeteners, water-soluble artificial sweeteners, water-soluble sweeteners derived from naturally occurring water-soluble sweeteners, dipeptide based sweeteners, and protein based sweeteners, including mixtures thereof. Without being limited to particular sweeteners, representative categories and examples include:
(a) water-soluble sweetening agents such as dihydrochalcones, monellin, steviosides, lo han quo, lo han quo derivatives, glycyrrhizin, dihydroflavenol, and sugar alcohols such as sorbitol, mannitol, maltitol, xylitol, erythritol, and L-aminodicarboxylic acid aminoalkenoic acid ester amides, such as those disclosed in U.S. Pat. No. 4,619,834, which disclosure is incorporated herein by reference, and mixtures thereof;
(b) water-soluble artificial sweeteners such as soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, and mixtures thereof;
(c) dipeptide based sweeteners, such as L-aspartic acid derived sweeteners, such as L-aspartyl-L-phenylalanine methyl ester (Aspartame), N-[N-(3,3-dimethylbutyl)-L-. alpha.-aspartyl]-L-phenylalanine 1-methyl ester (Neotame), and materials described in U.S. Pat. No. 3,492,131, L-alphaaspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, and mixtures thereof;
(d) water-soluble sweeteners derived from naturally occurring water-soluble sweeteners, such as chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro 1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1, 6-dichloro-1,6-dideo-xy-beta-D-fructofuranoside, or 4,6,1′,6′-tetrachloro-4,6, 1′,6′-tetradeoxygalacto-sucrose; and 4,6,1′,6′-tetradeoxy-sucrose, and mixtures thereof;
(e) protein based sweeteners such as thaumaoccous danielli (Thaumatin I and II) and talin; and
(f) the sweetener monatin (2-hydroxy-2-(indol-3-ylmethyl)-4-aminoglutaric acid) and its derivatives.

The intense sweetening agents may be used in many distinct physical forms well-known in the art to provide an initial burst of sweetness and/or a prolonged sensation of sweetness. Without being limited thereto, such physical forms include free forms, spray dried forms, powdered forms, beaded forms, encapsulated forms, and mixtures thereof. In one embodiment, the sweetener is a high intensity sweetener such as aspartame, sucralose, and acesulfame potassium (e.g., Ace-K or acesulfame-K).

In some embodiments, the sweetener may be a polyol. Polyols can include, but are not limited to glycerol, sorbitol, maltitol, maltitol syrup, mannitol, isomalt, erythritol, xylitol, hydrogenated starch hydrolysates, polyglycitol syrups, polyglycitol powders, lactitol, and combinations thereof.

In general, an effective amount of intense sweetener may be utilized to provide the level of sweetness desired, and this amount may vary with the sweetener selected. The intense sweetener may be present in amounts from about 0.001% to about 3%, by weight of the composition, depending upon the sweetener or combination of sweeteners used. The exact range of amounts for each type of sweetener may be selected by those skilled in the art.

### Food Acids

The pH of the composition is about 3 to about 5, about 3.7 to about 4. The pH may be adjusted by a food acid, buffer, or both.

Suitable food acids include but are not limited to acetic acid, adipic acid, ascorbic acid, butyric acid. citric acid, formic acid, fumaric acid, glyconic acid, lactic acid, phosphoric acid, malic acid, oxalic acid, succinic acid, tartaric acid, or combinations thereof.

Suitable buffers include but are not limited to sodium citrate and potassium citrate. For example, an acid/buffer system is 1.33% of a 54% citric acid solution, buffered with sodium citrate.

The amount of acid will be in the typical range of from about 0.5 to about 2% by weight, e.g., about 1.25%. Higher acid (lower pH) results in a lack of structure while lower acid levels do not provide enough “acid bite” in the flavor profile.

### Flavoring Agents

In some embodiments, the composition may further include a flavoring agent. Flavoring agents may include those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof.

In some embodiments, the flavoring agents may include mint(s), menthol, menthone, isomenthone, camphor and eucalyptol, eucalyptol, camphor, borneol, fenchone, menthone and isomenthone, isopulegol, monomenthyl succinate, and menthyl lactate, menthone, isomenthone, borneol, fenchone, eucalyptus, ducalyptol, ethyl benzoate, neomenthol, d-fenchone, furfurylidene butyrate, bucchu fractions, sage oil, corn mint oil, rosemary, monomenthyl succinate, amyl salicylate, eugenol, phellendrene, propyl furoate, ethyl-3-hydroxy butyrate, hexyl valerate, anisyl propionate, anysyl butyrate, dihydrocarveol, or clary sag.

Nonlimiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya and so forth. Other potential flavors whose release profiles can be managed include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide breath freshening properties, particularly the mint flavors when used in combination with the cooling agents, described herein below. In some embodiments, flavorants may chose from geraniol, linalool, nerol, nerolidal, citronellol, heliotropine, methyl cyclopentelone, ethyl vanillin, maltol, ethyl maltol, furaneol, alliaceous compounds, rose type compounds such as phenethanol, phenylacetic acid, nerol, linalyl esters, jasmine, sandlewood, patchouli, and/or cedarwood.

In some embodiments, other flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference. These may include natural as well as synthetic flavors.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, e.g., melonal (melon), 2,6-dimethyloctanal (green fruit), and 2-dodecenal (citrus, mandarin), cherry, grape, blueberry, blackberry, strawberry shortcake, and mixtures thereof.

In some embodiments, flavoring agents are used at levels that provide a perceptible sensory experience i.e. at or above their threshold levels. In other embodiments, flavoring agents are used at levels below their threshold levels such that they do not provide an independent perceptible sensory experience. At subthreshold levels, the flavoring agents may provide an ancillary benefit such as flavor enhancement or potentiation.

In some embodiments, a flavoring agent may be employed in either liquid form and/or dried form. When employed in the latter form, suitable drying means such as spray drying the liquid may be used. Alternatively, the flavoring agent may be absorbed onto water soluble materials, such as cellulose, starch, sugar, maltodextrin, gum arabic and so forth or may be encapsulated. In still other embodiments, the flavoring agent may be adsorbed onto silicas, zeolites, and the like.

In some embodiments, the flavoring agents may be used in many distinct physical forms. Without being limited thereto, such physical forms include free forms, such as spray dried, powdered, beaded forms, encapsulated forms, and mixtures thereof.

Illustrations of the encapsulation of flavors as well as other additional components can be found in the examples provided herein. Typically, encapsulation of a component will result in a delay in the release of the predominant amount of the component during consumption of a composition that includes the encapsulated component (e.g., as part of a delivery system added as an ingredient to the chewing composition). In some embodiments, the release profile of the ingredient (e.g., the flavor, sweetener, etc.) can be managed by managing various characteristics of the ingredient, delivery system containing the ingredient, and/or the composition containing the delivery system and/or how the delivery system is made. For example, characteristics might include one or more of the following: tensile strength of the delivery system, water solubility of the ingredient, water solubility of the encapsulating material, water solubility of the delivery system, ratio of ingredient to encapsulating material in the delivery system, average or maximum particle size of ingredient, average or maximum particle size of ground delivery system, the amount of the ingredient or the delivery system in the composition, ratio of different polymers used to encapsulate one or more ingredients, hydrophobicity of one or more polymers used to encapsulate one or more ingredients, hydrophobicity of the delivery system, the type or amount of coating on the delivery system, the type or amount of coating on an ingredient prior to the ingredient being encapsulated, etc.

### Sensate Agents

In some embodiments, the composition further includes a sensate agent. Sensate agents can include cooling agents, warming agents, tingling agents, effervescent agents, and combinations thereof. A variety of cooling agents may be employed. For example, among the useful cooling agents are included xylitol, erythritol, dextrose, sorbitol, menthane, menthone, ketals, menthone ketals, menthone glycerol ketals, Japanese mint oil, peppermint oil. These and other suitable cooling agents are further described in the following U.S. patents, all of which are incorporated in their entirety by reference hereto: U.S. Pat. Nos. 4,230,688; 4,032,661; 4,459,425; 4,136,163; 5,266,592; 6,627,233.

In some embodiments, warming components may be selected from a wide variety of compounds known to provide the sensory signal of warming to the user. These compounds offer the perceived sensation of warmth, particularly in the oral cavity, and often enhance the perception of flavors, sweeteners and other organoleptic components. In some embodiments, useful warming compounds can include vanillyl alcohol n-butylether, vanillyl alcohol n-propylether, vanillyl alcohol isopropylether, vanillyl alcohol isobutylether, vanillyl alcohol n-aminoether, vanillyl alcohol isoamyleather, vanillyl alcohol n-hexyleather, vanillyl alcohol methylether, vanillyl alcohol ethylether, gingerol, shogaol, paradol, zingerone, capsaicin, dihydrocapsaicin, nordihydrocapsaicin, homocapsaicin, homodihydrocapsaicin, ethanol, isopropyl alcohol, iso-amylalcohol, benzyl alcohol, glycerine, and combinations thereof.

In some embodiments, a tingling sensation can be provided. One such tingling sensation is provided by adding jambu, oleoresin, or spilanthol to some examples. In some embodiments, alkylamides extracted from materials such as jambu or sanshool can be included. Additionally, in some embodiments, a sensation is created due to effervescence. Such effervescence is created by combining an alkaline material with an acidic material. In some embodiments, an alkaline material can include alkali metal carbonates, alkali metal bicarbonates, alkaline earth metal carbonates, alkaline earth metal bicarbonates and mixtures thereof. In some embodiments, an acidic material can include acetic acid, adipic acid, ascorbic acid, butyric acid, citric acid, formic acid, fumaric acid, glyconic acid, lactic acid, phosphoric acid, malic acid, oxalic acid, succinic acid, tartaric acid and combinations thereof. Examples of “tingling” type sensates can be found in U.S. Pat. No. 6,780,443, the entire contents of which are incorporated herein by reference for all purposes.

Sensate agents may also be referred to as “trigeminal stimulants” such as those disclosed in U.S. Patent Application No. 205/0202118, which is incorporated herein by reference. Trigeminal stimulants are defined as an orally consumed product or agent that stimulates the trigeminal nerve. Examples of cooling agents which are trigeminal stimulants include menthol. WS-3, N-substituted p-menthane carboxamide, acyclic carboxamides including WS-23, methyl succinate, menthone glycerol ketals, bulk sweeteners such as xylitol, erythritol, dextrose, and sorbitol, and combinations thereof. Trigeminal stimulants can also include flavors, tingling agents, Jambu extract, vanillyl alkyl ethers, such as vanillyl n-butyl ether, spilanthol, Echinacea extract, Northern Prickly Ash extract, capsaicin, capsicum oleoresin, red pepper oleoresin, black pepper oleoresin, piperine, ginger olcoresin, gingerol, shoagol, cinnamon olcoresin, cassia oleoresin, cinnamic aldehyde, eugenol, cyclic acetal of vanillin and menthol glycerin ether, unsaturated amides, and combinations thereof.

In some embodiments, sensate agents are used at levels that provide a perceptible sensory experience i.e. at or above their threshold levels. In other embodiments, sensate components are used at levels below their threshold levels such that they do not provide an independent perceptible sensory experience. At subthreshold levels, the sensates may provide an ancillary benefit such as flavor or sweetness enhancement or potentiation.

### Freshening Agents

In some embodiments, the composition further includes a freshening agent. Freshening agents may include essential oils as well as various aldehydes, alcohols, and similar materials. In some embodiments, essential oils can include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, and orange. In some embodiments, aldehydes such as cinnamic aldehyde and salicylaldehyde can be used. Additionally, chemicals such as menthol, carvone, iso-garrigol, and anethole can function as breath fresheners. Of these, the most commonly employed are oils of peppermint, spearmint and chlorophyll.

In addition to essential oils and chemicals derived from them, in some embodiments freshening agent can include but are not limited to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based materials, carbon-based materials, enzymes such as laccase, and combinations thereof.

### Coloring Agents

Coloring agents may be used in amounts effective to produce the desired color. The coloring agents may include pigments which may be incorporated in amounts up to about 6%, by weight of the composition. For example, titanium dioxide may be incorporated in amounts up to about 2%, and preferably less than about 1%, by weight of the composition. The colorants may also include natural food colors and dyes suitable for food, drug and cosmetic applications. These colorants are known as F.D.& C. dyes and lakes. The materials acceptable for the foregoing uses are preferably water-soluble. Illustrative nonlimiting examples include the indigoid dye known as F.D.& C. Blue No. 2, which is the disodium salt of 5,5-indigotindisulfonic acid. Similarly, the dye known as F.D.& C. Green No. 1 comprises a triphenylmethane dye and is the monosodium salt of 4-[4-(N-ethyl-p-sulfoniumbenzylamino) diphenylmethylene]-[1-(N-ethyl-N-p-sulfoniumbenzyl)-delta-2,5-cyclohexadi-eneimine]. A full recitation of all F.D.& C. colorants and their corresponding chemical structures may be found in the Kirk-Othmer Encyclopedia of Chemical Technology, 3rd Edition, in volume 5 at pages 857-884, which text is incorporated herein by reference.

In some embodiments, one or more colors can be included. As classified by the United States Food, Drug, and Cosmetic Act (21 C.F.R. 73), colors can include exempt from certification colors (sometimes referred to as natural even though they can be synthetically manufactured) and certified colors (sometimes referred to as artificial), or combinations thereof. In some embodiments, exempt from certification or natural colors can include, but are not limited to annatto extract, (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), .beta.-apo-8′-carotenal (E160e), .beta.-carotene (E160a), alpha carotene, gamma carotene, ethyl ester of beta-apo-8 carotenal (E160f), flavoxanthin (E161a), lutein (E161b), cochineal extract (E120); carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), and combinations thereof.

In some embodiments, certified colors can include, but are not limited to, FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (E153), black PN/brilliant black BN (E151), green S/acid brilliant green BS (E142), and combinations thereof. In some embodiments, certified colors can include FD&C aluminum lakes. These include of the aluminum salts of FD&C dyes extended on an insoluble substrate of alumina hydrate. Additionally, in some embodiments, certified colors can be included as calcium salts.

In some embodiments, natural fruits or plant juice or extracts may be used as the coloring agents. Example include without limitation carrot juice, raspberry juice, blackberry juice, blueberry juice, and beet juice.

### Plasticizer

In some embodiments, the composition may further include plasticizer to modify the texture of the formulation. A texture modifying agent may include a particulate material. Suitable particulate materials can include, but are not limited to, sucrose, polyols such as sorbitol, xylitol, mannitol, galactitol, lactitol, maltitol, erythritol, isomalt, hydrogenated starch hydrolysates and mixtures thereof, starches, proteins, and combinations thereof. In some embodiments, the particulate material serving as a texture modifying component is selected based on its ability or lack of ability to crystallize the saccharides in the saccharide portion. For example, when isomalt is included in the saccharide portion, sorbitol powder can be added to the composition because it will not cause the isomalt to crystallize. Alternatively, when erythritol is included in the saccharide portion, erythritol powder can be added to the composition because it will cause the erythritol to crystallize. Such particulates can be included in amounts from 5% to 35% w/w of the composition.

### Fats and Oils

In some embodiments, a texture modifying component can include fats, oils, or other hydrophobic materials. Suitable fats can include, but are not limited to, partially hydrogenated vegetable or animal fats, such as coconut oil, corn oil, palm kernel oil, peanut oil, soy bean oil, sesame oil, cottonseed oil, cocoa butter, milk fat, beef tallow, and lard, among others. Suitable hydrophobic materials include chocolate, chocolate crumb, carob coatings, and compound coatings. Such fats, oils, and/or hydrophobic materials can be included in amounts of 1% to 10% w/w of the composition.

In some embodiments, the sensory perception of the texture modifying component is similar to that of fat, oil, or other hydrophobic materials. For example, a composition including 2.5% fats or oil can provide the same mouthfeel perception as a composition including 10%-50% fat as measured by sensory evaluation techniques.

Suitable oils and fats usable in compositions include vegetable or animal fats, such as butter, coconut oil, palm kernel oil, beef tallow, and lard, among others. These ingredients when used may be present in amounts up to about 7%, or up to about 3.5%, by weight of the composition.

In some embodiments, the composition may include edible oil component present in an amount of from about 1% to about 30%, alternatively 1% to 5%, alternatively 5% to 10%, alternatively 10% to 15%, alternatively 15% to 20%, alternatively 20% to 25%, alternatively 25% to 30%, by weight of the composition. In some embodiments, the edible oil component may be present in an amount of from about 0% to about 30%, alternatively 0% to 1%, alternatively 1% to 5%, alternatively 5% to 10%, alternatively 10% to 15%, alternatively 15% to 20%, alternatively 20% to 25%, alternatively 25% to 30%, by weight of the composition. This edible oil component makes up part of the group of ingredients that adjust the taste, texture, and improve the melt and mouth feel of the flavored chewy or gummy confection. For example, in some embodiments, the interaction of the group of highly unsaturated oils with the coconut oil component may create an improved elasticity within the flavored chewy confection that acts similar to hydrogenated or partially hydrogenated fat. The edible oil component also improves the health characteristic of the flavored chewy confection compositions because it adds monounsaturated and polyunsaturated fats. An example of an edible oil component is a blend of canola, soybean oil, and sunflower oil.

Non-limiting examples of edible oil components acceptable for use in the preferred embodiments include those that have low saturated fat content and high unsaturated fat including monounsaturated and especially polyunsaturated oils. The edible oil component should have no specific flavor and preferably is basically bland or somewhat buttery in taste. The edible oils component can be selected from the following; canola oil, soybean oil, safflower oil, sunflower oil, sesame oil, walnut oil, olive oil, flaxseed oil, chia seed oil, almond oil, corn oil, grape seed oil, peanut oil, other nut oils, and synthesized or reorganized oils, and combinations thereof.

In some embodiment, the edible oil component may have a high level of saturated fats present in an amount of from about 0.3% to about 20%, alternatively 0.3% to 3%, alternatively 3% to 5%, alternatively 5% to 10%, alternatively 10% to 15%, alternatively 15% to 20%, by weight of the composition. Suitable oils having a high level of saturated fats include, but are not limited to, one or more selected from the group consisting of coconut oil, palm oil, fractionated coconut or palm oil, partially hydrogenated coconut or palm oil, fully hydrogenated coconut or palm oil, or any other synthesized or altered edible oils including partially hydrogenated oils and fully hydrogenated oils that have either highly saturated or highly unsaturated fatty acids that when hydrogenated become solid similar to coconut oil in consistency including partially hydrogenated soybean oil, cotton seed oil, palm kernel oil or combination of these edible oils. In one embodiment, the edible oil component comprises coconut oil. This oil component forms a part of the flavor profile and provides a texture to the flavored chewy or gummy confection, and it improves the taste, texture, melt, and mouth feel of the compositions. The blend of the flavor components provides for a great taste, texture, melt and mouthfeel, without the necessity of using partially hydrogenated or fully hydrogenated oils. Any medium heat processed coconut oil can be used.

### Hemectant

The glycerin is a humectant and freezing point depressant. It also helps decrease the tendency for granulation and aid in maintaining softness. In some embodiment, glycerin or equivalent material may be employed at a level of from about 1 to about 5% by weight of the final product, e.g., 2 to 3%.

Humectants that can provide a perception of mouth hydration may be included. Such humectants can include, but are not limited to glycerol, sorbitol, polyethylene glycol, erythritol, and xylitol. Additionally, in some embodiments, fats can provide a perception of mouth moistening. Such fats can include medium chain triglycerides, vegetable oils, fish oils, mineral oils, and combinations thereof.

### Bulking Agents

Suitable sugar bulking agents include monosaccharides, disaccharides and polysaccharides such as xylose, ribulose, glucose (dextrose), lactose, mannose, galactose, fructose (levulose), sucrose (sugar), maltose, invert sugar, partially hydrolyzed starch and corn syrup solids, and mixtures thereof.

Suitable sugar alcohol bulking agents include sorbitol, xylitol, mannitol, galactitol, lactitol, maltitol, erythritol, isomalt and mixtures thereof. Suitable hydrogenated starch hydrolysates include those disclosed in U.S. Pat. No. 4,279,931 and various hydrogenated glucose syrups and/or powders which contain sorbitol, maltitol, hydrogenated disaccharides, hydrogenated higher polysaccharides, or mixtures thereof. Hydrogenated starch hydrolysates are primarily prepared by the controlled catalytic hydrogenation of corn syrups. The resulting hydrogenated starch hydrolysates are mixtures of monomeric, dimeric, and polymeric saccharides. The ratios of these different saccharides give different hydrogenated starch hydrolysates different properties. Mixtures of hydrogenated starch hydrolysates, such as LYCASIN.RTM., a commercially available product manufactured by Roquette Freres of France, and HYSTAR.RTM., a commercially available product manufactured by SPI Polyols, Inc. of New Castle, Del., are also useful.

### Emulsifiers

The composition may include an emulsifier. The emulsifier may present in an amount of from about 0.001% to about 5%, alternatively 0.001% to 1%, alternatively 1% to 3%, alternatively 3% to 5%, by weight of the composition. In some embodiments, the emulsifier present in an amount of from about 0% to about 5%, alternatively 0.001% to 1%, alternatively 1% to 3%, alternatively 3% to 5%, by weight of the composition.

Example emulsifiers include but not limited to modified corn starch, mono- and diglycerides, and lecithin.

The emulsifier may assist in holding together the fats and water and other components together in a homogeneous composition. In one embodiment, the emulsifier may assist in the formation of a “water and oil” emulsion that creates the smooth texture of the finished product.

### Preservatives

Preservatives may be natural or synthetic. Non-limiting examples of suitable preservatives include: sodium benzoate, sodium citrate, sodium phosphate, potassium metabisulfite, sodium metabisulfite, sodium lactate, sodium sulfite, EDTA (ethylenediaminetetraacetic acid), methylparaben, TBHQ, tocopherols, and mixtures thereof. Natural preservatives may include phenols (phenolic acid, polyphenols, tannins), isoflavonoids, organic acids (acetic, lactic, citric), and herb extracts such as extracts of citrus fruits, oregano, thyme, sage, rosemary, clove, coriander, garlic, and onion.

In some embodiments, the composition may include at least about 0% to 2%, by weight of the composition of a preservative component from above, or mixtures thereof.

### Liquids

Liquids may be used to assist in the flavoring and texture profile of the products. In some embodiments, the composition may include from about 0.001% to about 25% by weight of a fruit or vegetable or combination juice or concentrate component, alternatively 0.001% to 5%, alternatively 5% to 10%, alternatively 10% to 15%, alternatively 15% to 20%, alternatively 20% to 25%, by weight of the composition. The fruit or vegetable or combination juice or concentrate component adds a flavor to the flavored chewy or gummy confection. Any suitable source from the following may used in the embodiments; citrus fruit juices, orchard fruit juices, berry fruit juices, vine fruit juices, decolorized juices, and vegetable juices can be used for this component. The forms can come from juices or concentrates of fruits or vegetables.

In some embodiments, the composition may also include a water component present in an amount of about 0% or greater. The water component adds to the overall texture and melt and chewiness of the flavored chewy or gummy composition. For example, water may be used because of the increase in viscosity of some example compositions. In some embodiments, the composition may contain water from about 1% to about 20%, alternatively 1% to 5%, alternatively 5% to 10%, alternatively 10% to 15%, alternatively 15% to 20%, by weight of the composition.

### Thickening Agent

The composition may further include a thickening agent to help with the viscosity of the final product. Some thickening agents are gelling agents. Others act as mechanical thixotropic additives with discrete particles adhering or interlocking to resist strain.

In some embodiments, the thickening agent may be polysaccharides or protein. Example polysaccharides thickening agents include starches, vegetable gums and pectin. Example starch based thickening agents include arrowroot, cornstarch, katakuri starch, potato starch, sago, tapioca and their starch derivatives. Example vegetable gums based thickening agents may include alginin, guar gum, locust bean gum, and xanthan gum. Example protein based thickening agents include collagen, egg whites, furcellaran, and gelatin. Sugar based thickening agent may include agar and carrageenan.

### Gelling Components

The gelling compounds may include one or more gelling agents. A number of gelling agents may be utilized including without limitation, gelatin, pectin, gum Arabic, carrageenans, agar agar, high methoxy pectin, alginates, xanthan gum, locust bean gum, gellan gum, guar gum, modified or unmodified starches, cellulose gum, modified starch wheat flour or enriched wheat flour or bleached flour or any type of flour from a natural source, or a combination thereof. Other example gelling agents may include acacia, alginic acid, bentonite, Carbopols® (now known as carbomers), carboxymethyl cellulose, ethylcellulose, gelatin, hydroxyethyl cellulose, hydroxypropyl cellulose, magnesium aluminum silicate (Veegum®), methylcellulose, poloxamers (Pluronics®), polyvinyl alcohol, sodium alginate, and tragacanth.

The amount of gelling agents used in the composition depends upon the texture, viscosity and softness of a desired product as well as other ingredients in the composition. In some embodiments, the gelling agents may be used in concentrations of about 0.5% to about 10%, about 0.1% to about 7%, or about 0.2 to about 15%.

In one embodiment, the gelling component may include pectin, gellan gum, carrageenans, alginate, or starch to provide a gelatin-free composition.

In one embodiment, the electrolyte hydration composition may include 2.7%-4%, 3%-3.9%, or 3.2%-3.7% of pectin. In one embodiment, the electrolyte hydration composition may include from about 0.25% to about 0.75% by weight gellan gum and about 2% to about 3% by weight.

In one embodiment, pectin at 3%-3.8% by weight produces a gummy composition with TPA hardness values in excess of 13 lbs (f), and TPA cohesiveness and elasticity values of 60% to 72%.

In one embodiment, a combination of gellan gum at about 0.25% to about 0.75% by weight and carrageenans at about 2.5% to about 3% by produces a gummy composition with TPA hardness values from about 12 to about 15 lbs (f), and TPA cohesiveness and elasticity values of 30% to 50%.

### Texture Profile Analysis (TPA)

Texture Profile Analysis (TPA) is used to characterize the aspects of the texture of a gelled or gummy product. Specifically, TPA cohesiveness is a measure of the resiliency of a product, TPA elasticity is a measure of springiness, and TPA hardness is a measure of firmness. For example, a gelatin gel tends to have high resiliency and high springiness, along with relatively high firmness.

The embodiments now will be described in more detail with reference to the non-limiting examples that follow.

### EXAMPLES

The following examples are provided to illustrate the preparation of gummy confections in accordance with this invention and are provided by way of illustration and are not intended to limit the invention in any way.

### Example. Electrolyte Gummy for Hydration

Ingredients: glucose syrup, sucrose, fructose, maltose, zinc citrate, magnesium oxide, sodium chloride, potassium chloride, citric acid, pectin, coconut oil, lemon oil, stevia.

Combine water, a first mixture of carbohydrates, and salts and heat until brix 70-95 to provide a first mixture.

Combine pectin and a second mixture of carbohydrates and water. Heat to provide a gelling mixture.

Combine the first mixture and the gelling mixture, and add citric acid, color, flavoring, and sweetener. Heat the mixture until brix 93 to provide a gummy batter.

The gummy batter was then added to silicone molds and allowed to cool to room temperature. Within an hour the gelation occurred.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.
While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An electrolyte hydration composition, comprising,
a mineral composition from about 2.5% to about 7% w/w, wherein the mineral component comprises salts of sodium, potassium, magnesium, or zinc,
a carbohydrate composition from about 70% to about 75%, and
a gelling component in a sufficient amount to provide a cohesive gelled product.

2. The electrolyte hydration composition of claim 1, wherein the mineral composition comprises salts or chelates of sodium, potassium, magnesium, zinc, calcium, phosphorus, or a combination thereof.

3. The electrolyte hydration composition of claim 2, wherein the chelates are amino acid chelates or peptide chelates.

4. The electrolyte hydration composition of claim 1, comprising at least 3% by weight of the mineral composition.

5. The electrolyte hydration composition of claim 1, wherein the carbohydrate comprises glucose, maltose, trehalose, maltodextrin, starch, glucose syrup, maltose syrup, or a combination thereof.

6. The electrolyte hydration composition of claim 1, further comprises a vitamin composition, an amino acid composition, an antioxidant composition, an herbal composition, and an anti-inflammatory composition.

7. The electrolyte hydration composition of claim 1, wherein the gelling component comprises gelatin, starch, pectin, gellan gum, gum Arabic, carrageenans, guar, agar, alginate, locust bean gum, xanthan, or derivatives thereof.

8. The electrolyte hydration composition of claim 1, wherein the gelling component comprises at least 3.5% of apple pectin having a DE of not less than 55%.

9. The electrolyte hydration composition of claim 1, wherein the gelling component comprises at least 3.2% of high methoxy citric pectin having DE of not less 40%.

10. The electrolyte hydration composition of claim 1, further comprises a food acid, wherein the food acid comprises malic acid, fumaric acid, lactic acid, tartaric acid, glucono-delta lactone, salts of gluconic acid, phosphoric acid, succinic acid, adipic acid, acetic acid, citric acid, or a combination thereof.

11. The electrolyte hydration composition of claim 1, wherein the pH of the composition is about 2.7 to about 3.7.

12. The electrolyte hydration composition of claim 1, comprising from about 69% to about 73% of the carbohydrate composition, from about 3% to about 6% of the mineral composition, from about 3.4% to about 4.5% of the gelling component, wherein the gelling component comprises a pectin having DE not less than 45%.

13. The electrolyte hydration composition of claim 12, wherein the pectin is an apple pectin.

14. The electrolyte hydration composition of claim 12, further comprises about 0.1%- 1% of calcium pantothenate.

15. The electrolyte hydration composition of claim 12, wherein the carbohydrate composition 40 comprises trehalose, maltose, or a combination thereof.

16. The electrolyte hydration composition of claim 12, wherein the carbohydrate composition comprises glucose syrup having a DE of not more than 24, maltose, or a combination thereof.

17. The electrolyte hydration composition of claim 12, wherein the carbohydrate composition comprises glucose syrup having a DE of not more than 42, trehalose, or a combination thereof.

18. A process for preparing an electrolyte hydration composition, comprising,
preparing a first premix by heating a mixture of ingredients comprising a sweetener, a gelling component, and water sufficiently to dissolve in water.
preparing a gelling solution by heating a carbohydrate composition and a mineral composition in water sufficiently to dissolve in water; and
blending the first premix and the gelling solution to form a combined mixture.
